(19) United States
(12) Patent Application Publication
(10) Pub. No.: US 2020/0221955 A1
(43) Pub. Date: Jul. 16, 2020

(54) METHODS AND SYSTEMS FOR USING NEAR INFRARED SPECTROSCOPY TO DETECT COMPARTMENT SYNDROME

(71) Applicant: UNIVERSITY OF FLORIDA RESEARCH FOUNDATION, INC., Gainesville, FL (US)

(72) Inventors: Patrick J. TIGHE, Gainesville, FL (US); Nikolaus GRAVENSTEIN, Gainesville, FL (US); Andre Pierre BOEZAART, Gainesville, FL (US); Sean A. FRITH, Gainesville, FL (US); Alina Zare GLENN, Newberry, FL (US); Sanjeev Jagannatha KOPPAL, Gainesville, FL (US)

(21) Appl. No.: 16/609,087

(22) PCT Filed: Sep. 10, 2018
(86) PCT No.: PCT/US2018/050196

§ 371 Date: Oct. 28, 2019

Related U.S. Application Data
(60) Provisional application No. 62/559,314, filed on Sep. 15, 2017.

(51) Int. Cl. A61B 5/00

(52) U.S. Cl. CPC A61B 5/0073; A61B 5/0075; A61B 5/14551; A61B 5/745; A61B 2562/0233; A61B 5/7278

(57) ABSTRACT

According to a method for using near infrared spectroscopy to detect compartment syndrome, an optics array is placed around a body region of a patient that contains a vulnerable compartment of tissue. The optics array includes emitters that generate photons at NIR wavelengths and detectors that detect photons at NIR wavelengths. Photons are emitted from at least one of the emitters, and detected at more than one of the detectors after those photons traveled through the tissues of the body region of the patient. A cross-sectional measure of regional differences in absorption within a cross-section of the body region of the patient is displayed so as to allow detection of compartment syndrome. The method is carried out with a device for using near infrared spectroscopy to detect compartment syndrome.

### STATEMENT REGARDING FEDERALLY SPONSORED RESEARCH OR DEVELOPMENT

This invention was made with government support under R01 GM114290 awarded by the National Institutes of Health. The government has certain rights in the invention.

### FIELD OF THE DISCLOSURE

The disclosure relates to near infrared spectroscopy, and more specifically to the use of near infrared spectroscopy to create a tomographic reconstruction for the detection of compartment syndrome.

### BACKGROUND

Compartment syndrome is a serious medical condition in which increased pressures within closed regions of the body can lead to serious complications if left untreated. Both acute and chronic compartment syndrome are caused by an extreme increase in the intracompartmental pressure of a closed osteofascial compartment. Acute compartment syndrome (ACS) is most commonly caused by a fractured bone. Other causes of ACS include soft tissue damage without fractures and substantial vascular injuries. Studies have shown that mortality rates can be 47% in patients with a severe case of ACS. Although the average incidence rate is relatively low (1 to 7.3 per 100,000), the severity of each case necessitates extra attention to timely identification of this condition.

Typically following traumatic injuries to a limb, ACS is caused by an excess of blood within the tissues with inadequate venous outflow. This causes elevated pressure within the affected compartment which can lead to collapsed lymphatic vessels, muscle necrosis, infection, permanent neurological damage, and often even limb amputation. In as little as 30 minutes after the injury, the onset of specific nerve symptoms can occur. Once ACS has been diagnosed, it is extremely important for an immediate intervention as irreversible damage can occur in hours. ACS is a serious condition that requires a quick fasciotomy intervention to prevent necrosis, permanent nerve and muscle loss, and amputation.

Current methods of diagnosis are limited in accuracy and require an invasive procedure. In cases in which the risk of developing ACS is high, physicians must routinely monitor patients for the five “P's” associated with the condition. Of these, pain, paresthesia, pallor, and paralysis are all non-specific symptoms determined through subjective assessments made in standard patient examinations. Conventionally, high intra-compartment pressure (a pressure of 20 mmHg to 45 mmHg or higher is an indicator of ACS) can only be determined by a hand-held needle manometer or a wick and slit catheter. These conventional devices are painful and invasive, and not very accurate. The inaccuracy in diagnosis combined with the seriousness of the condition can lead to surgery being performed on patients that do not even have ACS, resulting in pain and loss of function.

Because a well-timed fasciotomy can prevent the most serious complications, improving and optimizing the diagnosis technologies would drastically improve patient care and treatment. In particular, what is needed is a new, non-invasive, and more accurate system and method for diagnosing compartment syndrome.

### SUMMARY OF THE DISCLOSURE

One aspect of the present invention relates to a method for using near infrared spectroscopy to detect compartment syndrome. According to the method, an optics array is placed around a body region of a patient that contains a vulnerable compartment of tissue. The optics array includes emitters that generate photons at near infrared (NIR) wavelengths (as well as in the vicinity of NIR wavelengths such as red and IR wavelengths) and detectors that detect photons at NIR wavelengths (again as well as in the vicinity of NIR wavelengths such as red and IR wavelengths). Photons are emitted from at least one of the emitters, and detected at one or more of the detectors after those photons traveled through the tissues of the body region of the patient. A cross-sectional measure of regional differences in absorption within a cross-section of the body region of the patient is displayed so as to allow detection of compartment syndrome.

The disclosure also relates to a device that uses near infrared spectroscopy to detect compartment syndrome. The device includes emitters that generate photons at NIR wavelengths, detectors that detect photons at NIR wavelengths, and an optics array capable of being wrapped around a body region of a patient that contains a compartments of tissue(s). The emitters and detectors are located in the optics array. The device also includes a signal receiver and a display. The signal receiver receives from the optics array data relating to photons detected by the detectors after those photons were generated by the emitters and traveled through the tissues of the body region of the patient, and the display displays a cross-sectional measure of regional differences in absorption within a cross-section of the body region of the patient so as to allow detection of compartment syndrome.

Embodiments described herein may provide a device for using at least one of infrared or near infrared (NIR) spectroscopy to detect actual or impending compartment syndrome. The device may include: a plurality of emitters that generate photons at NIR wavelengths; a plurality of detectors that detect photons at NIR wavelengths; and optics array capable of being wrapped around a body region of a patient that contains a plurality of distinct compartments of tissue, the emitters and detectors being located in the optics array; a signal receiver receiving, from the optics array, data relating to photons detected by the detectors in response to those photons being generated by the emitters and emitted through tissue of the body region of the patient; a signal processor to determine, based on photon propagation, tissue structures and boundaries thereof that define the plurality of distinct compartments of tissue; and a display for displaying a cross-sectional measure of regional differences in absorption within the distinct compartments of tissue in a cross-section of the body region of the patient so as to allow detection of compartment syndrome.

According to some embodiments, the cross-sectional measure of regional differences depicts estimated pressures and/or oxygen saturation levels of the plurality of distinct compartments of tissue based on the data received from the optics array and an inverse algorithm. The inverse algorithm may be a three-dimensional modeling algorithm, and the display displays a tomographic representation of the distinct compartments of tissue reconstructed from the received data using the three-dimensional modeling algorithm. The tomographic representation may show a cross section depicting the relative oxygenation levels of the tissue in each of the distinct compartments of tissue of the body region of the patient. The tomographic representation is based, at least in part, upon differing absorption spectra of oxygenated/deoxygenated hemoglobin. The tomographic representation may further be based, at least in part, upon differing absorption spectra from myoglobin and other tissues.

The device of some embodiments may include emitters that are light emitting diode (LED) or Laser Diode emitters, and the detectors are photomultiplier tubes, silicon p-i-n photodiodes, or avalanche photodiodes. The emitters and the detectors of the optics array are evenly spaced in a single ring. The emitters and the detectors of the optics array may be arranged in a mesh both radially and longitudinally defining a substantially cylindrical shape.

Embodiments provided herein may include a method for using at least one infrared or near infrared (NIR) spectroscopy to detect compartment syndrome. Methods may include: placing an optics array around a body region of a patient that contains a plurality of distinct compartments of tissues, the optics array including a plurality of emitters that generate photons at NIR wavelengths and a plurality of detectors that detect photons at NIR wavelengths; emitting photons from at least one of the emitters; detecting photons at more than one of the detectors in response to those photons being emitted by the at least one emitter and having traveled through the body region of the patient; processing the detected photons to determine, based on photon propagation, tissue structures and boundaries thereof that define the plurality of distinct compartments of tissue, and displaying a cross-sectional measure of regional difference in absorption within the distinct compartments of tissue in a cross-section of the body region of the patient so as to allow detection of compartment syndrome.

According to some embodiments, the cross-sectional measure depicts estimated pressures and/or oxygen saturation levels of the plurality of distinct compartments of tissue based on an inverse algorithm. The inverse algorithm may be a three-dimensional modeling algorithm, and displaying the cross-sectional measure comprises displaying a tomographic representation of the plurality of distinct compartments of tissue that is reconstructed using the three-dimensional modeling algorithm. The tomographic representation may show a cross section depicting the relative oxygenation levels of each of the plurality of compartments of tissue of the body region of the patient. Methods may include continuously updating the tomographic representation to indicate a trending of the relative oxygenation levels of the plurality of compartments of tissue.

The tomographic representation may be based, at least in part, upon differing absorption spectra of oxygenated/deoxygenated hemoglobin. The tomographic representation may further be based, at least in part, on differing absorption spectra from myoglobin and other tissues. The emitting and detecting operations may include emitting photons from a first of the emitters and detecting those photons at all of the detectors; after emitting photons from the first emitter, emitting photons from a second of the emitters and detecting those photons at all of the detectors; and after emitting photons from the second emitter, sequentially repeating the emitting and detecting for the remaining emitters so as to serially activate all of the emitters. The second emitter may be adjacent to the first emitter and the sequential repeating may include activating adjacent emitters. In some embodiments, the second emitter may not be adjacent to the first emitter. After sequentially repeating the emitting and detecting so as to serially activate all of the emitters, methods may repeat the emitting and detecting process so as to continuously serially activate all of the emitters.

### BRIEF DESCRIPTION OF THE DRAWINGS AND PICTURES

A more complete understanding of the present disclosure, and the attendant advantages and features thereof, will be more readily understood by reference to the following detailed description when considered in conjunction with the accompanying drawings wherein:

FIG. 1 shows a cross-section of the four compartments of the lower leg;

FIG. 2 is a plot of the reflectance properties of tissues for a light pulse;

FIG. 3 shows the results of a Monte Carlo modeling for the transport of a single photon in a homogenous medium;

FIG. 4 shows the results of Monte Carlo modeling for photon propagation in heterogeneous tissues with varying oxygenation saturation;

FIG. 5 shows an emitter/detector mesh schematic for a NIRS imaging device according to one embodiment of the present invention;

FIG. 6 shows a block diagram of the NIRS imaging device of FIG. 5;

FIG. 7 shows multiple detectors detecting light from a single emitter in the NIRS imaging device of FIG. 5;

FIGS. 8-12 show a method for using near infrared spectroscopy to detect compartment syndrome according to one embodiment of the present invention;

FIG. 13 shows the use of a conventional oxygen probe, modeling the behavior of a conventional oxygen probe when applied to tissues with overlapping/concentric differences in anticipated oxygenation status;

FIG. 14 shows the use of conventional NIRS methods for tissue oximetry; and

FIG. 15 shows multiple detectors detecting light from a single emitter in a NIRS imaging device according to an embodiment of the present invention.

### DETAILED DESCRIPTION

As required, embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

Embodiments of the present invention use near infrared spectroscopy (NIRS) to create a tomographic reconstruction of a body region for detecting compartment syndrome.

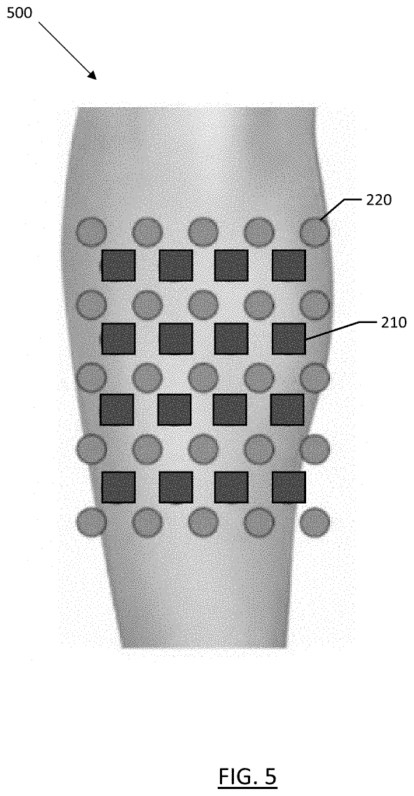

One embodiment of the invention provides a NIRS device that can monitor oxygen saturation across a region of the lower leg where ACS often manifests. Experiments conducted show differences in anticipated photon propagation across the tissue types of the lower leg, and principles of tomographic reconstruction are used to solve this problem. The device includes a ring of IR/NIR emitters and detectors that encircles a tissue at risk for compartment syndrome. Emitters and detectors are arranged in an alternating pattern at very close intervals. This can be extended to a mesh that also extends the adjacency proximal and distal, in addition to the circular adjacency described (FIG. 5).

The device transmits IR and NIR light, and associated relevant wavelengths, from a single emitter and detects at all other emitters. Then, the device repeats this single emission and multi-site detection with another emitter. This is repeated until the device has cycled through the activation of all emitters. In some embodiments, the device then keeps continuously cycling through the emitters as needed. The device uses computed tomography of the IR/NIR/related wavelengths to report a cross-sectional measure of regional differences in absorption within the cross-section of the body region under the device, so as to allow detection of compartment syndrome (e.g., through changes of hemoglobin concentration, saturation, and relative saturation within discrete regions and compartments within the limb) within overlapping compartments.

This allows for earlier and more accurate detection of compartment syndrome before severe or permanent tissue injury occurs. It does so in a noninvasive manner and allows for continuous assessment, even through casts and splints in some embodiments. This device is much better for detecting ACS than just placing a conventional oxygen saturation (SpO2) probe on a digit. A conventional transmission oxygen saturation probe emits two or three wavelengths of light and measures change in absorbance at each wavelength. Such a probe requires a thin section of body for accuracy. A conventional reflectance oxygen saturation probe uses reflectance to determine arterial oxygen saturation. Such a probe would be overwhelmed by normal oxygen saturation levels in other compartments. As a result, both types of conventional oxygen saturation probes therefore have very poor sensitivity and specificity for detecting compartment syndrome.

The NIRS device of the present invention is also much better for detecting ACS than known somatic tissue NIRS-based monitoring systems that measure oxygen levels in the brain and other vital organs. Such systems use NIR light but only sample superficial tissue (e.g., 1-2 cm under the skin) and cannot differentiate which tissues are oxygenated at which levels. Therefore, such systems are unable to assess deep/overlapping compartments as is necessary for detecting compartment syndrome. And the NIRS device of the present invention is clearly advantageous over conventional approaches that require insertion of a needle/catheter into the affected tissues. Besides being invasive, such approaches have limited effectiveness in detecting compartment syndrome, require accurate placement and calibration by trained surgical teams, and offer only late recognition of adverse oxygen delivery to affected compartments.

An exemplary embodiment using NIRS for creating a tomographic reconstruction of the lower leg region to detect ACS will now be described. One popular pathophysiological theory for ACS is that circulation of blood from arteries to veins is obstructed with a diminished pressure gradient between the two circulation systems. This not only decreases the rate at which oxygenated blood enters the tissue, but also decreases the venous drainage of deoxygenated blood. This creates a feedback loop in which intracompartmental pressure rises and total oxygen saturation decreases. This change in oxygenation allows NIRS to be used as a diagnostic tool for physicians to diagnose ACS. Additionally, even in absence of changes in oxygenated/deoxygenated hemoglobin, this technique can specify total light absorption by hemoglobin within specific locations that can indicate early venous congestion, hematoma formation, and other pathophysiologic states associated with hemoglobin aggregation.

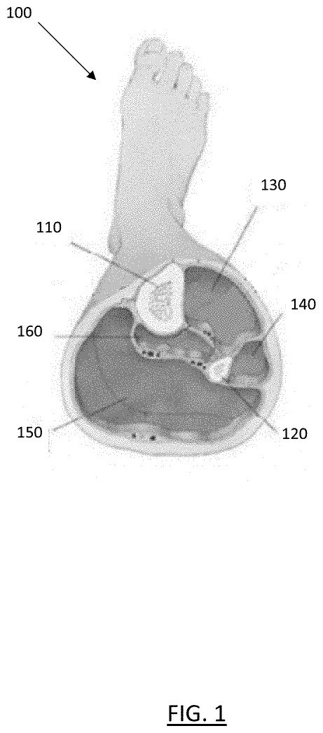

FIG. 1 shows a cross-section of the four distinct compartments and boundaries thereof of the lower leg 100, as well as the positions of the tibia 110 and fibula 120. As shown, the lower leg has four muscle compartments: anterior 130, lateral 140, superficial posterior 150, and deep posterior 160. All the different tissues in the lower leg have varying optical properties, and NIRS takes advantage of these differences in light propagation to collect relevant physical data such as oxygenation saturation. NIRS utilizes near-infrared light with wavelengths ranging from 650-950 nm. The photons of this light travel relatively deep into tissues and their differential absorption and scattering depends on the optical properties which include the local oxygen saturation. To understand this scattering of photons within tissues, tissue scattering and absorption have been accurately separated by directly modeling the diffusive process. The concepts of scattering length and transport mean-free path are directly relative to the absorption constant and scattering constant properties of tissues.

To use NIRS to diagnose ACS, both the “forward-problem” and the “inverse-problem” must be solved with respect to imaging the lower leg. The “forward-problem” involves modeling the way in which the photons propagate through different types of tissues. For this purpose, photon propagation in varying tissue structures was studied to determine the ways in which light emitted from a NIRS device will traverse through a variety of tissue types and tissue structures with varying levels of oxygen saturation.

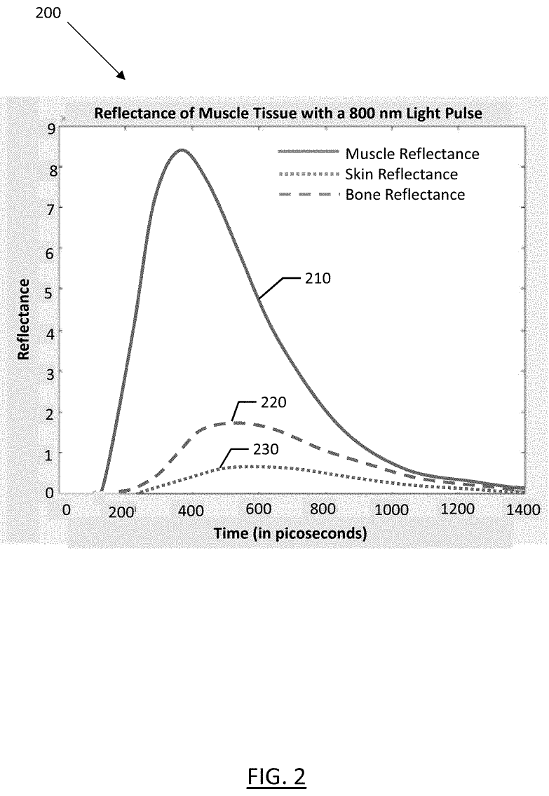

A single-photon Monte Carlo experiment was performed to show how the physical equations guide light propagation. Monte Carlo modeling works backwards from the result based on known probabilities to determine how the result could have been produced. In particular, the reflectance properties of different tissues with respect to NIR light were studied using MATLAB software. This modeling incorporated key optical properties such as the transport scattering coefficient and absorption coefficient. A list of the constants used is shown in Table 1. The modeling utilized the diffusion equation, the kappa equation, and the logarithmic reflectance equation shown below. These equations utilize the absorption and scattering coefficients for each tissue type. FIG. 2 is a plot 200 of these equations modeling the reflectance properties of tissues for a single 800 nm light pulse over 1400 picoseconds. Line 210 represents muscle reflectance, line 220 represents bone reflectance, while line 230 represents skin reflectance.

D=1/[3(μ′ₛ+μₐ)]

κ=−1.5*log(4πv₁D)−log(μₐ+μ′ₛ)

R=κ−2.5*log(t)−[v₁t+(3ρ²/4v₁t)]μₐ−(3ρ²/4v₁t)μ′ₛ

TABLE 1
Constants

| Constant | Value |
|---|---|
| C (speed of light) | 3.00 × 10⁸ |
| n (tissue scattering constant) | 1.4 |
| ν₁ (speed of light in tissue) | c/n |
| ρ (source detector separation | 20 mm |
| distance) | |
| μ′ₛ (transport scattering coefficient) | Bone = 1.6; skin = 1.9; |
| | muscle = 1.0 |
| μₐ (absorption coefficient) | Bone = 0.016; skin = |
| | 0.018; muscle = 0.017 |

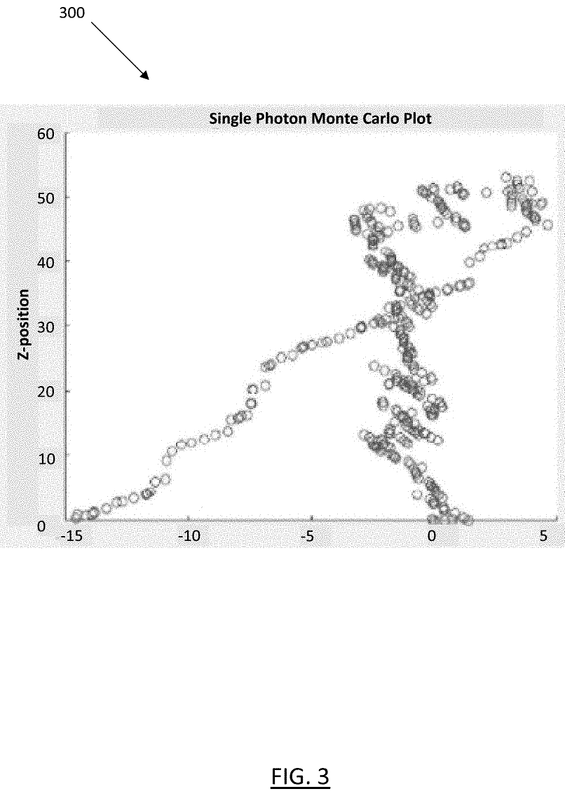

Another Monte Carlo experiment was conducted to show the transport of a single photon of NIR light in a homogenous medium. This provides a better understand of the physics of NIR/IR light propagation before performing a full Monte Carlo experiment in a realistic tissue phantom. Using a host of equations and variables, a MATLAB script was produced that simulated a photon traveling through tissues producing absorption or scattering events at each location. Energies were calculated and boundary conditions were considered. The results of this experiment are shown in plot 300 of FIG. 3. As shown, the photon had an initial position at the origin (0,0) and an initial direction toward (0,0,1). Based on random probabilities and the physics of tissue optics, step sizes were calculated along with direction changes. When the result is full attenuation within the tissue, the simulation is halted. Otherwise the simulation continues until the photon successfully escapes the external boundary.

Because this simulation could only handle a single photon, a more robust Monte Carlo software package was used to model the way that NIRS beams would traverse through different tissue types. This more elaborate Monte Carlo modeling was carried out using the software package known as “mcxyz.c” (available from the Oregon Medical Laser Center) due to its ability to model photon propagation in heterogeneous tissues. A MATLAB script was used to designate the optical properties of different tissue types as well as to design the tissue phantoms for the model. This software was used to model many packets of photons (e.g., 200), rather than a single photon as in the previous experiments.

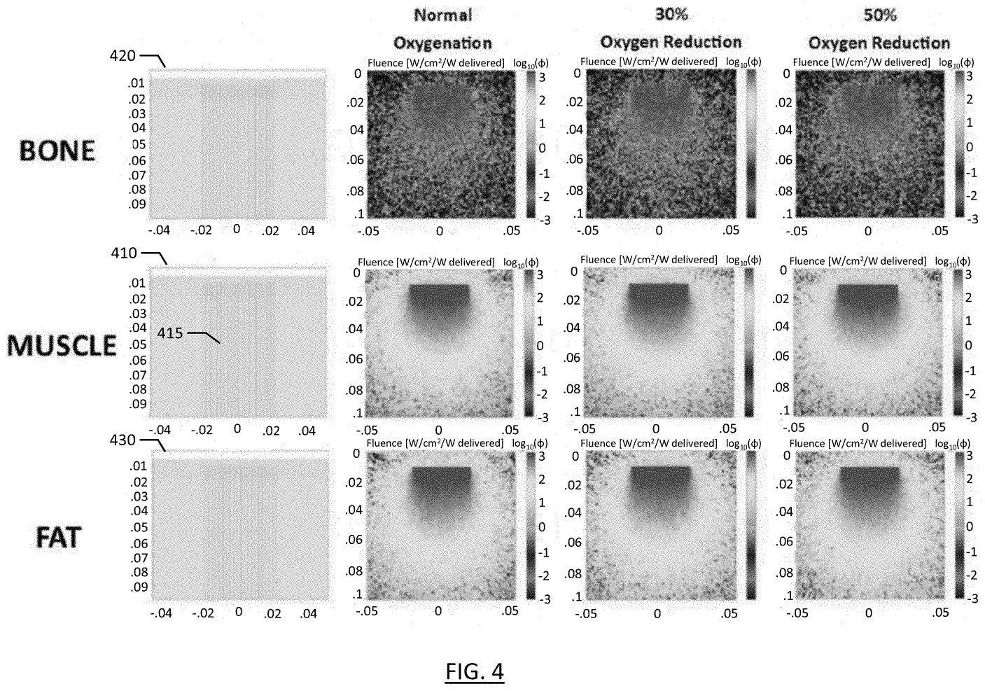

The middle row leftmost plot 410 of FIG. 4 shows an exemplary skin/muscle tissue phantom. This phantom contains a skin dermis layer and a muscle layer. The vertical lines 415 indicate the NIRS photons entering the tissue and where they would travel without scattering events. The upper and lower leftmost plots 420, 430 of FIG. 4 similarly show a skin/bone tissue phantom model and a skin/fat tissue phantom model. Table 2 shows the optical properties used for each tissue type in this modeling. All three tissue types (i.e., muscle, bone, and fat) were studied with varying levels of oxygen saturation: standard saturation, a 30% decrease in saturation, and a 50% decrease in saturation.

The other plots of FIG. 4 show the results of the Monte Carlo modeling for the tissue type of each row at varying oxygenation saturation values. In particular, the Monte Carlo modeling produced fluence rate plots, with the fluence rate value indicating the number of photon particles crossing a point per unit time (which correlates directly to the measured photons at a receptor). This gives a general understanding of the depth penetration in a variety of tissue types that are encountered in the lower leg region. The three tissue types (i.e., muscle, fat, and bone) were chosen for modeling because they are the primary types of tissues found in the lower leg region. Because the depth of penetration is shallow and the emitters will only be directed toward compartments of muscle tissue, homogenous tissue composition can be assumed.

TABLE 2
Tissue Optical Properties

| Tissue | Variable | Value |
|---|---|---|
| Skin | BVF (Blood Volume | 0.002 |
| (Dermis) | Fraction) | |
| | S (Oxygen Saturation | 0.67 |
| | of Hemoglobin) | |
| | W (Water Volume | 0.65 |
| | Fraction) | |
| | Musp (Reduced | 42.4 |
| | Scattering Coefficient) | |
| Bone | BVF | 0.0005 |
| | S | 0.75 |
| | W | 0.35 |
| | Musp | 30 |
| Muscle | BVF | 0.1 |
| | S | 0.75 |
| | W | 0.75 |
| | Musp | 20 |
| Fat | BVF | 0.1 |
| | S | 0.67 |
| | W | 0.29 |
| | Musp | 20 |

These Monte Carlo experiments showed the subtle differences found across types of tissues and tissues with varying oxygen saturation levels. Although the depth of penetration is relatively low, NIRS imaging allows a large region of tissue to be imaged at this shallow depth. Because three out of the four compartments have regions near the skin surface, the compartment pressures can be determined from the oxygen saturation levels of the shallow tissue regions.

The “inverse-problem” with respect to imaging the lower leg involves selecting the most suitable method of tomographic reconstruction for lower-leg imaging. For this purpose, the Levenberg-Marquaardt algorithm or another suitable algorithm can be used to solve the inverse problem and create a reconstruction matrix for identification of the absorption coefficient within the tissues being imaged.

A NIRS imaging device according one embodiment of the present invention includes the following functionality. The device detects and measures relative saturations of the lower leg compartments using NIRS technology, and displays the results to the medical staff. The device does this while performing continuous monitoring of the lower leg region. In this embodiment, the device displays the results shows by showing a tomographic image representation of the lower leg region to the medical staff, and also producing an output that shows the precise compartment that is pressurized (if any). Preferably, the device is small enough that it does not obstruct movement in the patient's room.

The NIRS imaging device of this embodiment also includes the following design characteristics. In the example of the leg embodiment the device fits around the patient's leg at the trauma site, without impeding access to the trauma site. The device operates with the patient in a normal position and does not impact blood flow to the lower leg region. Preferably, the device is easily sterilized or has disposable patient contact sites.

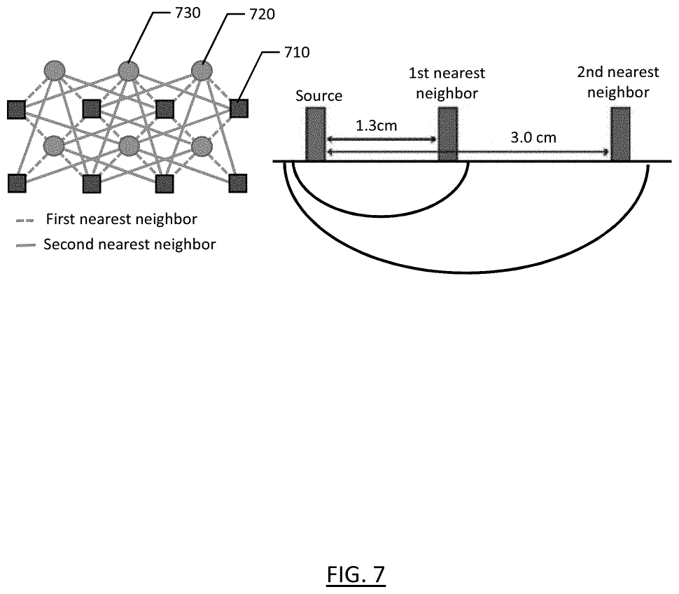

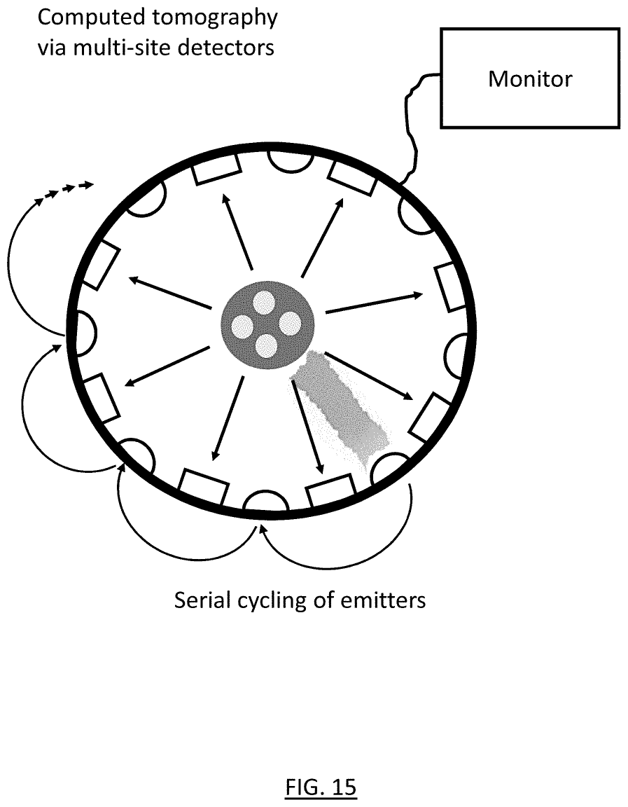

FIG. 5 shows the emitter/detector mesh schematic 500 of the NIRS imaging device of this embodiment. The red squares 210 indicate emitter locations and the blue circles 220 indicate detector locations. To acquire image data for the leg compartments, the mesh of emitters and detectors are wrapped around the patient's lower leg (i.e., the targeted leg region). The emitters are evenly spaced at a distance of 2-4 cm from one another, and the receptors are also evenly spaced away from one another at the same distance. As shown in FIGS. 7 and 15, this mesh layout provides multiple “neighbor” detectors for detecting the light from a single emitter. In other words, each emitter is part of multiple emitter-detector pairs at varying distances. As shown in FIG. 7, an array of emitters represented as squares including emitter 710 and detectors represented as circles including 720 and 730 provide a network of emitter/detector pairs. The first nearest neighbor is the closest emitter to a given detector while a second nearest neighbor is a next closest emitter to a given detector. As shown, multiple emitter/detector pairs may be the first nearest neighbor as in the illustrated mesh the first nearest neighbor distances are equal or substantially equal. The emitter or source may emit photons which are then received by the first and second nearest neighbors. As shown, a source emitter 710 may emit a photon which is received at the first nearest neighbor 720 at 1.3 centimeters away, while photons from the source 710 may also be received at the second nearest neighbor 730 at 3.0 centimeters away. This mesh produces many source-detector measurements that allow data to be reconstructed through a three-dimensional (3D) modeling algorithm to show the oxygen saturation in specific leg regions. For example, the measurements can be supplied to a Monte Carlo simulation that has been modified to work with NIR/IR light to produce a tomographic representation of the leg regions. The tomographic reconstruction shows a cross section that includes the relative oxygenation levels of tissues concentrically arranged. In another embodiment, the mesh is replaced with a simpler ring structure.

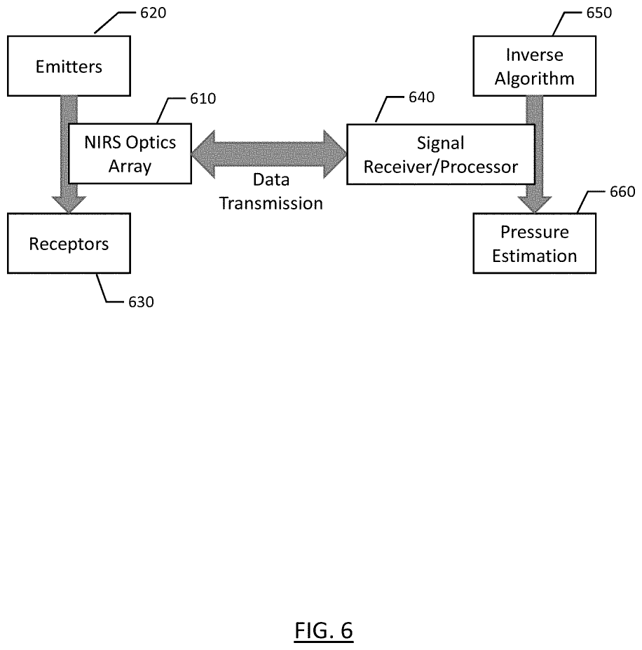

FIG. 6 shows a block diagram of the NIRS imaging device of this embodiment, including primary communication pathways. As shown, a NIRS optics array 610 includes the emitters 620 and detectors/receptors 630. The emitters generate photons at NIR wavelengths (and optionally also at IR and/or other related wavelengths), and the receptors detect these photons after they have traveled through the patient's tissues. The NIRS optics array transmits data relating to these detections to a signal receiver/processor 640. The signal receiver/processor receives the data from the NIRS optics array 610 and processes the signal using an inverse algorithm 650, the results of which are used to make a pressure estimation 660.

Embodiments of the present invention can use different types of NIR emitters, such as LED or Laser Diode (LD) emitters. LED emitters have the advantages of being small, inexpensive, and easy to adjust, while also offering a greater variation of wavelengths, increased emission into tissue, and minimal power consumption. However, LED emitters have a lower optical power output to consumption ratio. LD emitters have the advantages of higher quality signal generation, sharp peaks, and higher intensities. However, LD emitters are larger, higher heat, higher cost, have higher safety demands, and offer less wavelength customization. The NIRS imaging device of FIGS. 5 and 6 uses LED emitters due to the increased penetration depth required for diagnosing the lower leg compartments. The use of LED emitters also offers lower costs with many emitters being used to emit photons throughout the targeted lower leg region in this embodiment. In particular, this exemplary device uses 16 LED emitters available from Vishay Semiconductor under product ID VSMY98145DSCT.

Likewise, embodiments of the present invention can use different types of NIR detectors, such as photomultiplier tubes, silicon p-i-n photodiodes, or avalanche photodiodes. Photomultiplier tubes have the advantages of very high sensitivity, large gains, and high speed. However, photomultiplier tubes and very large and bulky, and require high voltage and cooling. Silicon p-i-n photodiodes have the advantages of small size, high dynamic range, strong resistance to ambient light exposure, and are easy to use. However, silicon p-i-n photodiodes have low sensitivity, reduced SNR and bandwidth, and do not provide internal amplification. Avalanche photodiodes have the advantages of being moderately small, resistant to ambient light exposure, and offer higher sensitivity. However, avalanche photodiodes require high voltages and cooling. The NIRS imaging device of FIGS. 5 and 6 uses silicon p-i-n photodiodes due to their small size and ability to operate without extremely high voltages. Other detectors are used in embodiments that require higher sensitivity. In particular, this exemplary device uses 25 silicon p-i-n photodiodes available from Texas Instruments under product ID OPT101P.

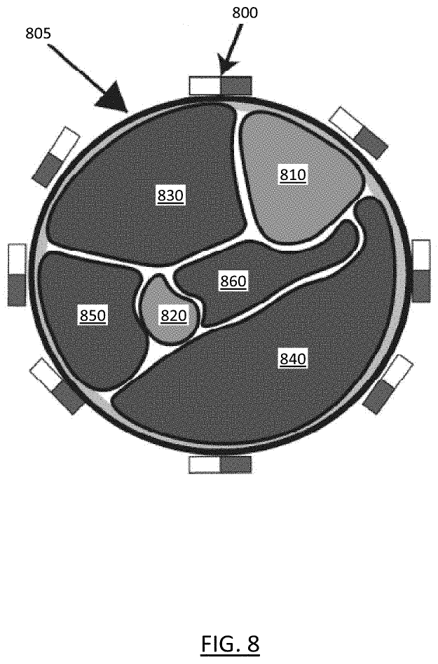

A method for using near infrared spectroscopy to detect compartment syndrome will now be described with reference to FIGS. 8-12. As shown in FIG. 8, the NIRS device used in this embodiment has a single ring of evenly spaced pairs of light emitter/detectors 800 connected via a continuous cable connection at 805 that is attached to either an elastic or fixed circular structure about the leg of a patient. As shown, the light emitter/detector pairs 800 surround the distinct compartments of tissue including the tibia 810, the fibula 820, the anterior compartment 830, the superficial posterior compartment 840, the lateral compartment 850, and the deep posterior compartment 860. In further embodiments, other patterns are used for the emitters and detectors such as volumetric extensions (in which the emitter/detectors are arranged both radially and longitudinally along a cylinder), random spacing, and/or separate emitters and detectors. A cable conducts power and data from the sensors to a control unit with a display. The device is fit around a lower leg that is at risk for ACS (or a related disease involving impaired tissue oxygenation, such as isolated vascular insufficiency or chronic compartment syndrome).

The tibia and fibula are shown along with the four compartments that contain muscle, nerve, and vascular tissues. These compartments overlap, whether considered via medial/lateral, radial, or anterior/posterior arrangements. In compartment syndrome, a compartment becomes ischemic (or even necrotic) while adjacent compartments retain sufficient oxygen delivery. The differential absorptions, reflectance, and scattering of light by oxygenated and deoxygenated hemoglobin, myoglobin, and other structures are complicated by the overlapping nature of the compartments. The device samples along a plethysmograph at both high and low frequency alternating currents. In further embodiments, the device samples mixed tissues without alternating currents.

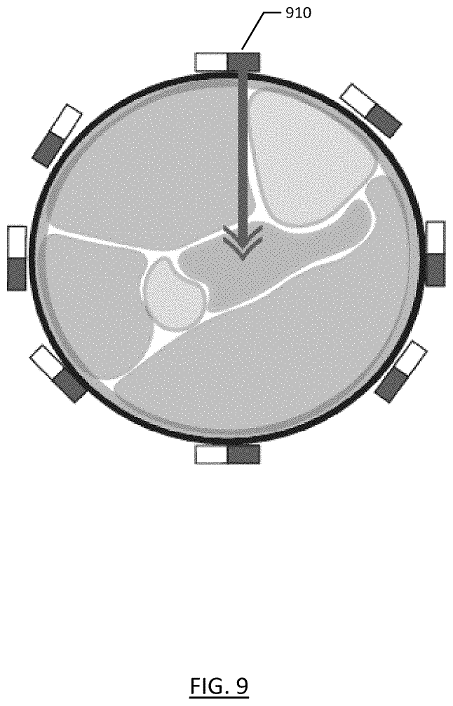

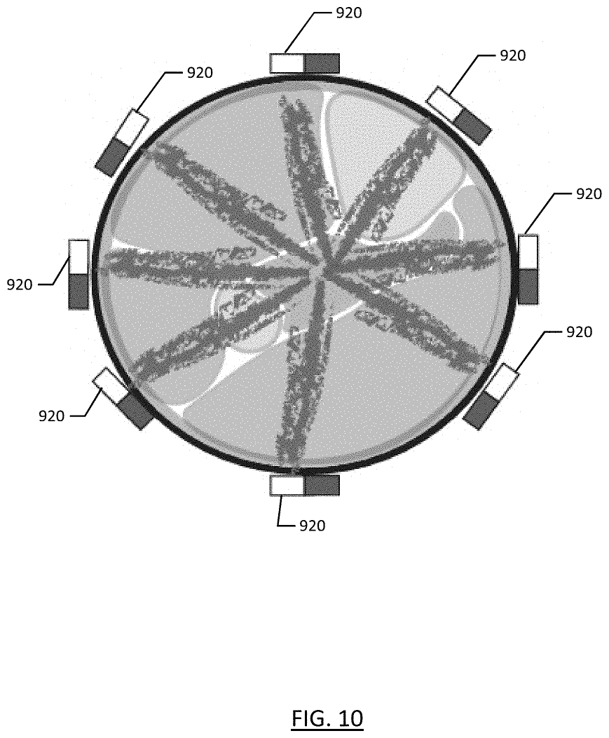

The process begins with emission of IR/NIR/related wavelength light from a single emitter source 910, as shown in FIG. 9. Following emission of the photon packet from the emitter, emission pauses to allow time for the photons to be transmitted, absorbed, reflected, and received at the circumferential detectors 920, as shown in FIG. 10. While this is described as a process of emission followed by detection, the rapidity of the process may approximate a continuous function. And in further embodiments, targeted simultaneous emissions from multiple emitters are used to enhance the quality and/or speed of the tomographic representation.

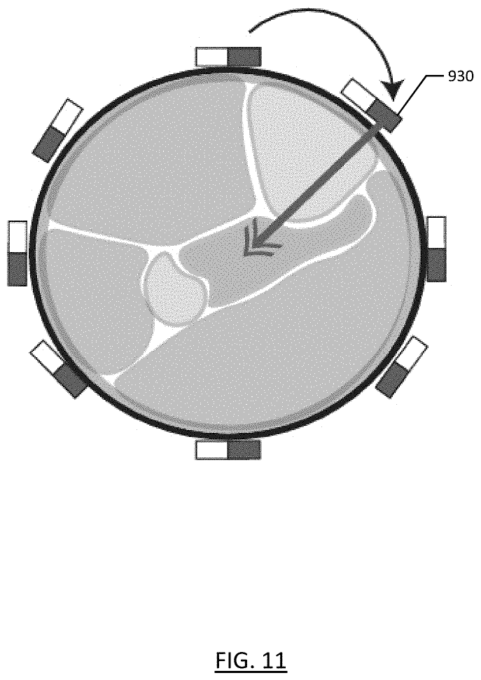

Following the initial emission-detection step, the device cycles to the next adjacent emitter and repeats the single-emitter/multi-detector process, at 930 as shown in FIG. 11. This cycling from the active emitter to the next adjacent emitter is repeated so as to serially activate all of the emitters in the ring. Due to variance and noise, multiple complete cycles through the emitters may be necessary to obtain sufficient information to create the tomographic representation. In further embodiments, the emitters are serially activated in a different patterned sequence or in a random sequence.

Thus, there is rapid, serial cycling of single (and/or simultaneous) emitters, followed by detection at all detectors. As the cycle progresses through the sequence of emitters, each detector will observe differences, both in relation to one another and in relation to previous emission cycles, in absorption, spectra, detection, time of flight, scatter, and so on. The different detection patterns are based upon origin of emission and characteristics of tissues and their absorption/scatter behavior, which is in part defined by the relative oxygenation status of venous and arterial hemoglobin as well as myoglobin and related proteins.

A tomographic representation is then reconstructed using a 3D modeling algorithm. In this embodiment, the tomographic representation is updated continuously to indicate trending of relative oxygenation status of the compartments. The tomographic representation of this embodiment is based upon the differing absorption spectra of oxygenated/deoxygenated hemoglobin, as well as the differing absorption spectra from myoglobin and other tissues across a range of wavelengths. In some embodiments, Bayesian priors of underlying anatomy, time-of-flight considerations of photon packets, and other machine learning methods are used to facilitate the tomographic reconstruction.

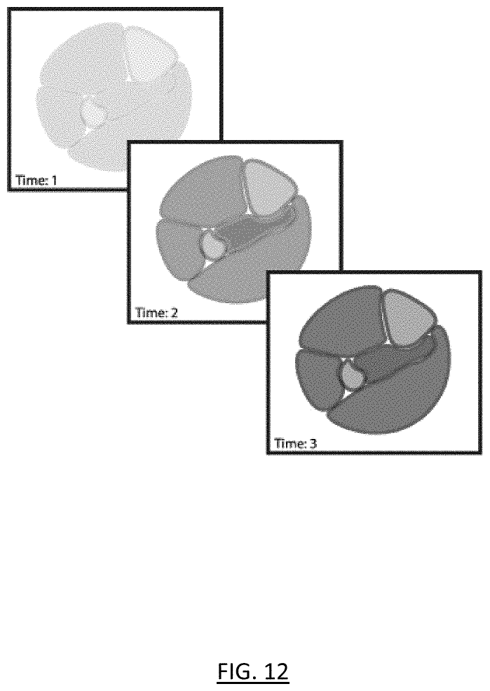

FIG. 12 shows an example of a tomographic representation visualized over time to indicate trending of developing compartment syndrome. This tomographic reconstruction shows a cross section depicting the relative oxygenation levels of the tissues in each compartment. Such a tomographic representation over time allows the relative deoxygenation to be detected before significant ischemia and death of compartmentalized tissues. Thus, earlier intervention is possible to preserve function and decrease local and systemic adverse events.

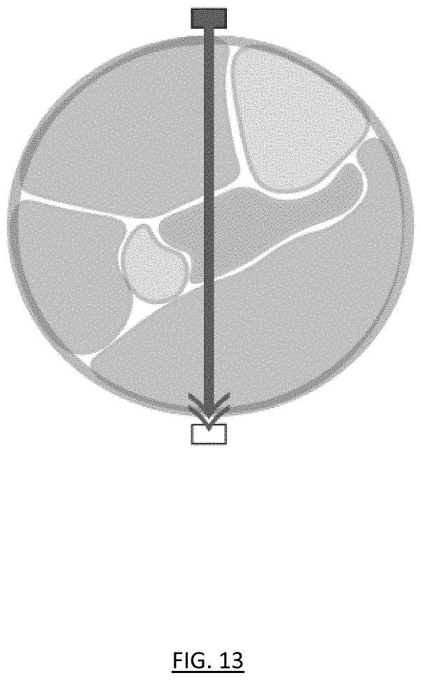

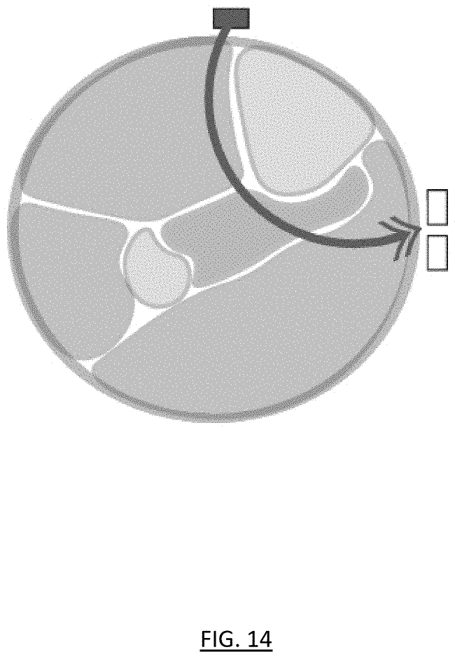

Thus, the devices and methods of the present invention use tomographic representation to permit determination of compartment syndrome in an environment having ‘hidden’, overlapping compartments (such as the deep posterior compartment of the lower leg). In contrast, when used on overlapping compartments, a conventional oxygen probe having a single emitter and a single detector for transmission-based oximetry experiences signal contamination from healthy tissues that overlap the ischemic compartment (see FIG. 13). Similarly, conventional NIRS methods for tissue oximetry, even those with multiple detectors, rely on reflectance oximetry and also experience signal contamination from overlapping healthy tissues (see FIG. 14).

Accordingly, embodiments of the present invention provide devices and methods for using near infrared spectroscopy to create a tomographic reconstruction of a body region to detect compartment syndrome. The device is non-invasive, low power, and low heat, so as to allowing for placement under dressings or even using sterile equipment for tissues at-risk of infection. The device is low cost and does not utilize ionizing radiation (unlike other more intensive imaging modalities). Furthermore, the device is small enough to complement existing diagnostic machines in operating, emergency, or patient rooms. Thus, embodiments of the present invention provide a non-invasive alternative to conventional pressure monitoring devices.

The NIRS devices and methods of the present invention are particularly suited for use in detecting compartment syndrome in the lower leg. However, the NIRS devices and methods of the present invention are also applicable for detecting compartment syndrome in the upper leg, the upper or lower arm, or even the trunk, chest, or skull of a patient.

And while the above description describes devices and methods in which the output of each emitter is assessed by multiple detectors, this is for illustrative purposes only and the present invention is not so limited. The present invention encompasses many emitter/detector combinations, including embodiments in which the output of each emitter is assessed by a single detector (which may or may not be the closest detector), embodiments in which the output of each emitter is assessed by multiple but not all detectors (which may or may not be the closest detector), and embodiments in which the output of each emitter is assessed all detectors.

Further, the detectors of the device may be permanent or disposable, and can surround the affected limbs for prolonged durations of time. The device can be fixed in structure or elastic, in order to minimize external pressure on affected limbs.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Additionally, an embodiment of the present invention may not include all of the features described above. Accordingly, all expedient modifications readily attainable by one versed in the art from the disclosure set forth herein that are within the scope and spirit of the present disclosure are to be included as further embodiments of the present disclosure.

What is claimed is:

1. A device for using at least one of infrared or near infrared (NIR) spectroscopy to detect actual or impending compartment syndrome, said device comprising:
a plurality of emitters that generate photons at NIR wavelengths;
a plurality of detectors that detect photons at NIR wavelengths;
an optics array capable of being wrapped around a body region of a patient that contains a plurality of distinct compartments of tissue, the emitters and detectors being located in the optics array;
a signal receiver receiving, from the optics array, data relating to photons detected by the detectors in response to those photons being generated by the emitters and emitted through tissue of the body region of the patient;
a signal processor to determine, based on photon propagation, tissue structures and boundaries thereof that define the plurality of distinct compartments of tissue; and
a display for displaying a cross-sectional measure of regional differences in absorption within the distinct compartments of tissue in a cross-section of the body region of the patient so as to allow detection of compartment syndrome.

2. The device of claim 1, wherein the cross-sectional measure depicts estimated pressures and/or oxygen saturation levels of the plurality of distinct compartments of tissue based on the data received from the optics array and an inverse algorithm.

3. The device of claim 2, wherein the inverse algorithm is a three-dimensional modeling algorithm, and the display displays a tomographic representation of the distinct compartments of tissue reconstructed from the received data using the three-dimensional modeling algorithm.

4. The device of claim 3, wherein the tomographic representation shows a cross section depicting the relative oxygenation levels of the tissue in each of the distinct compartments of tissue of the body region of the patient.

5. The device of claim 4, wherein the tomographic representation is based, at least in part, upon differing absorption spectra of oxygenated/deoxygenated hemoglobin.

6. The device of claim 5, wherein the tomographic representation is further based, at least in part, upon differing absorption spectra from myoglobin and other tissues.

7. The device of claim 1, wherein the emitters are light emitting diode (LED) or Laser Diode emitters, and the detectors are photomultiplier tubes, silicon p-i-n photodiodes, or avalanche photodiodes.

8. The device of claim 1, wherein the emitters and the detectors of the optics array are evenly spaced in a single ring.

9. The device of claim 1, wherein the emitters and the detectors of the optics array are arranged in a mesh both radially and longitudinally defining a substantially cylindrical shape.

10. A method for using at least one of infrared or near infrared (NIR) spectroscopy to detect compartment syndrome, said method comprising:
placing an optics array around a body region of a patient that contains a plurality of distinct compartments of tissues, the optics array including a plurality of emitters that generate photons at NIR wavelengths and a plurality of detectors that detect photons at NIR wavelengths;
emitting photons from at least one of the emitters;
detecting photons at more than one of the detectors in response to those photons being emitted by the at least one emitter and having traveled through the body region of the patient;
processing the detected photons to determine, based on photon propagation, tissue structures and boundaries thereof that define the plurality of distinct compartments of tissue; and
displaying a cross-sectional measure of regional differences in absorption within the distinct compartments of tissue in a cross-section of the body region of the patient so as to allow detection of compartment syndrome.

11. The method of claim 10, wherein the cross-sectional measure depicts estimated pressures and/or oxygen saturation levels of the plurality of distinct compartments of tissue based on an inverse algorithm.

12. The method of claim 11,
wherein the inverse algorithm is a three-dimensional modeling algorithm, and
displaying the cross-sectional measure comprises displaying a tomographic representation of the plurality of distinct compartments of tissue that is reconstructed using the three-dimensional modeling algorithm.

13. The method of claim 12, wherein the tomographic representation shows a cross section depicting the relative oxygenation levels of each of the plurality of compartments of tissue of the body region of the patient.

14. The method of claim 13, further comprising continuously updating the tomographic representation to indicate trending of the relative oxygenation levels of the plurality of compartments of tissue.

15. The method of claim 13, wherein the tomographic representation is based, at least in part, upon differing absorption spectra of oxygenated/deoxygenated hemoglobin.

16. The method of claim 15, wherein the tomographic representation is further based, at least in part, upon differing absorption spectra from myoglobin and other tissues.

17. The method of claim 10, wherein the emitting and detecting comprises:
emitting photons from a first of the emitters and detecting those photons at all of the detectors;
after emitting photons from the first emitter, emitting photons from a second of the emitters and detecting those photons at all of the detectors; and
after emitting photons from the second emitter, sequentially repeating the emitting and detecting for the remaining emitters so as to serially activate all of the emitters.

18. The method of claim 17, wherein the second emitter is adjacent to the first emitter, and the sequential repeating comprises sequentially activating adjacent emitters.

19. The method of claim 17, wherein the second emitter is not adjacent to the first emitter.

20. The method of claim 17, wherein after sequentially repeating the emitting and detecting so as to serially activate all of the emitters, repeating the emitting and detecting process so as to continuously serially activate all of the emitters.
